(19) United States
(12) Patent Application Publication
(10) Pub. No.: US 2019/0153376 A1
(43) Pub. Date: May 23, 2019

(54) MULTIWELL PLATE WITH INTEGRATED STIRRING MECHANISM

(71) Applicant: The Regents of the University of California, Oakland, CA (US)

(72) Inventors: Elliot En-Yu Hui, Irvine, CA (US); Erik Morgan Werner, Irvine, CA (US); Philip N. Duncan, Irvine, CA (US); Transon V. Nguyen, Irvine, CA (US); Siavash Ahrar, Irvine, CA (US)

(21) Appl. No.: 16/254,835

(22) Filed: Jan. 23, 2019

Related U.S. Application Data
(63) Continuation-in-part of application No. 15/711,946, filed on Sep. 21, 2017, now abandoned.
Application No. 15/711,946 is a continuation-in-part of application No. 14/029,286, filed on Sep. 17, 2013, now Pat. No. 9,784,258.
(60) Provisional application No. 61/813,099, filed on Apr. 17, 2013.
(60) Provisional application No. 61/702,709, filed on Sep. 18, 2012.

(51) Int. Cl. C12M 1/02; F04B 43/12; C12M 3/06; C12M 1/32; C12M 1/00; C12M 1/34; C12M 1/36

(52) U.S. Cl. CPC C12M 27/00; F04B 43/1207; C12M 23/16; F04B 2207/02; C12M 29/14; C12M 41/40; C12M 41/48; C12M 23/12

(57) ABSTRACT

This invention describes a design for a multiwell plate that contains integrated pumps that are used to stir each well of the plate. The device employs microfluidic logic technology to drive each peristaltic pump. This enables the plates to run autonomously, requiring only a static vacuum supply for power. The devices are entirely constructed out of low-cost polymers, with no electronics, and yet contains simple digital logic circuits to control the pumps. A stack of these plates may be run continuously in a standard cell culture incubator, allowing high-throughput culture of organoids.

### CROSS-REFERENCES TO RELATED APPLICATIONS

This application is a continuation-in-part and claims benefit of U.S. patent application Ser. No. 15/711,946, filed Sep. 21, 2017 which is a continuation-in-part of U.S. Non-Provisional application Ser. No. 14/029,286 filed Sep. 17, 2013, now U.S. Pat. No. 9,784,258, which claims benefit to U.S. Provisional Applications 61/702,709 filed Sep. 18, 2012 and 61/813,099 filed Apr. 17, 2013, the specification(s) of which is/are incorporated herein in their entirety by reference.

### STATEMENT REGARDING FEDERALLY SPONSORED RESEARCH OR DEVELOPMENT

This invention was made with government support under Grant No. ECCS-1102397, awarded by the National Science Foundation (NSF); Grant No. N66001-10-1-4003, awarded by the Space and Naval Warfare Systems Command (SPAWAR). The government has certain rights in the invention.

### BACKGROUND OF THE INVENTION

Human brain organoids are three-dimensional cultured tissues formed out of pluripotent stem cells. These constructs are useful for studying neural development and brain disorders, and they are currently attracting great interest in the stem cell community. Preparation of organoids requires culture in a continuously stirred suspension culture, but the use of stir bars and flasks results in low-throughput.

Flasks with magnetic stir bars are large and bulky, thus sharply constraining the throughput of organoids that can be cultured at once and the number of different culture conditions that can be tested in parallel. The use of motorized propeller arrays is able to reduce the culture volume and increase the throughput, however current systems are limited to 12-well plates, and it is unclear whether this approach can be scaled much further to higher density plates. In addition, the number of plates that can be run in parallel is limited by the physical size of the mechanical system (currently roughly the size of 4-5 plates) as well as the number of propeller systems available.

### Field of the Invention

The present invention relates to microfluidic devices for biological culturing. More specifically, the present invention relates to multiwell plates which include an integrated microfluidic stirring mechanism and are configured for the culture of brain organoids.

### Description of Related Art Including Information Disclosed

See attached IDS.

### BRIEF SUMMARY OF THE INVENTION

It is an objective of the present invention to provide devices and methods that allow for the stirring of a plurality of wells on a multiwell plate, as specified in the independent claims. Embodiments of the invention are given in the dependent claims. Embodiments of the present invention can be freely combined with each other if they are not mutually exclusive.

The present invention features a multiwell plate that contains integrated peristaltic pumps that are used to stir each well of the plate. The device employs microfluidic logic technology to drive each peristaltic pump. This enables the plates to run autonomously, requiring only a static vacuum supply for power. The devices may be entirely constructed out of low-cost polymers, with no electronics, and yet contain simple digital logic circuits to control the pumps. A stack of these plates may be run continuously in a standard cell culture incubator, allowing high-throughput culture of organoids.

The multiwell plates of the present invention combine standard format microtiter plates with an array of microfluidic logic oscillator pumps. For each well of the microtiter plate, cell culture media may be drawn from the well and pumped back into the well by one or more peristaltic pumps to create fluid jets that impart convective flow to the media. The jets may be angled to stir and agitate the media in various flow patterns, including but not confined to rotational motion. The shape and velocity of the convective flow patterns may be tuned experimentally for optimal organoid culture.

This approach employs specially designed microfluidic pumps that may be fabricated with very small dimensions and may allow higher densities such as 96-well plates. In addition, the pumps and controls are integrated into the plates themselves, which may be no larger than a standard plate, making it feasible to run large numbers of plates in parallel. Each plate will require only a single pneumatic connection to supply a static vacuum for power. The house vacuum that is widely available across biology laboratories may be sufficient to power the system.

One of the unique and inventive technical features of the present invention is the use of microfluidic logic technology and peristaltic pumps which are integrated within a microfluidic plate. Without wishing to limit the invention to any theory or mechanism, it is believed that the technical feature of the present invention advantageously provides for the stirring of a large number of culture wells on a plate which requires only a single pneumatic connection to a static vacuum for power. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1A shows a schematic drawing of an integrated multiwell stirring plate of the present invention.

FIG. 1B shows a schematic drawing of a pump system of the present invention.

FIG. 2A shows a diagram of a pneumatic oscillator circuit with three inverter logic gates.

FIG. 2B shows a diagram of an oscillator pump, including a three-inverter ring oscillator circuit coupled with three in-line fluid valves for peristaltic pumping of fluids from a fluid inlet through the three fluid valves to a fluid outlet.

FIG. 3 shows a graphical representation of the output values at nodes 1, 2, and 3 of FIG. 2B and a graphical and diagrammatic representation of the opening and closing of valves A, B, and C of FIG. 2B as a function of time.

FIG. 4A shows a diagram of a pneumatic membrane valve of the present invention in which the valve is in the closed position, with the membrane in a default position.

FIG. 4B shows a diagram of a pneumatic membrane valve of the present invention in which the valve is in the opened position, with the membrane in a deformed position.

FIG. 5 shows an expanded-view diagram of a pneumatic membrane valve of the present invention.

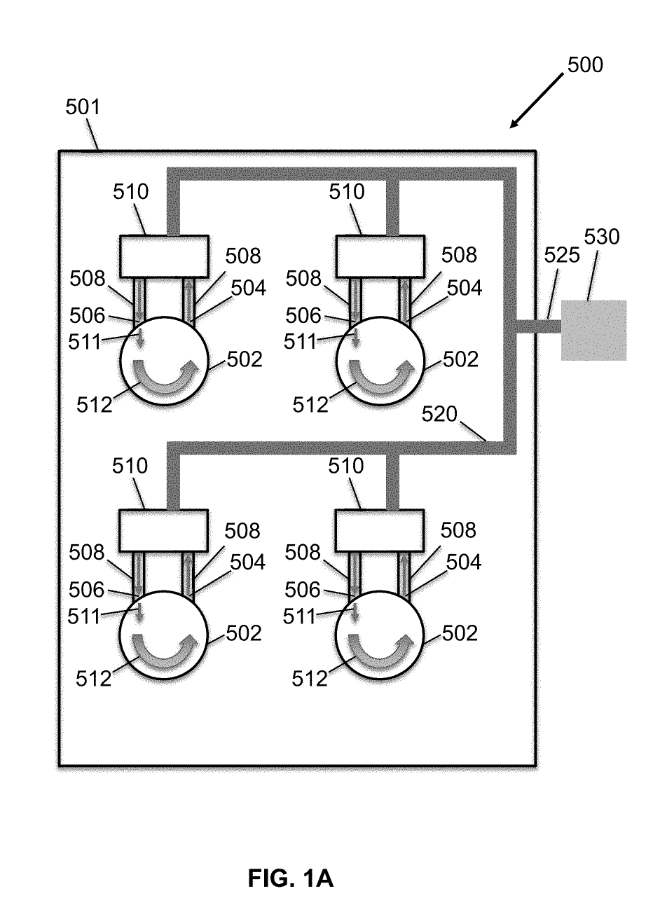

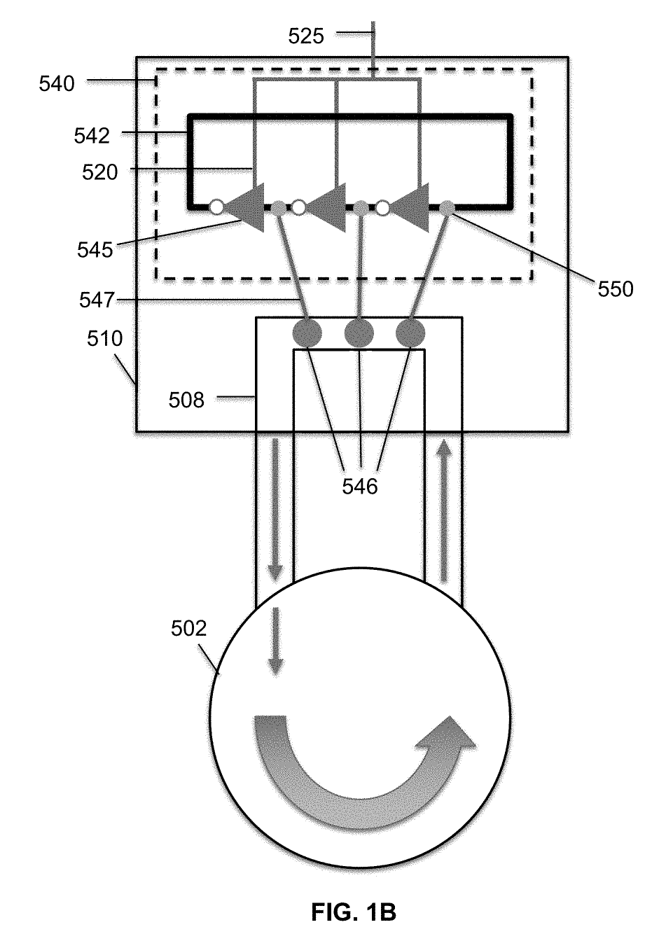

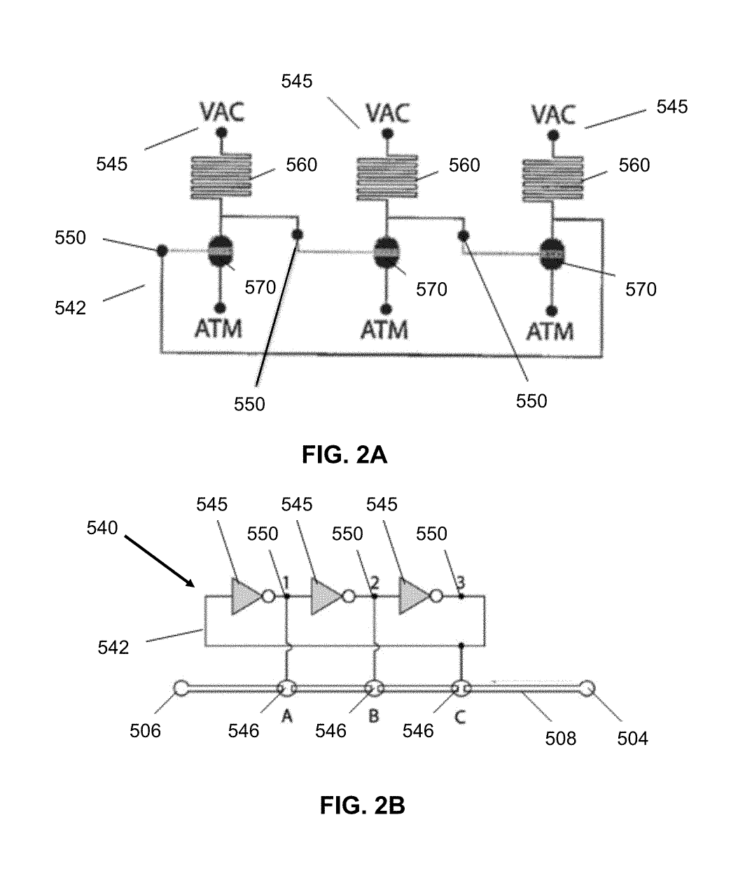

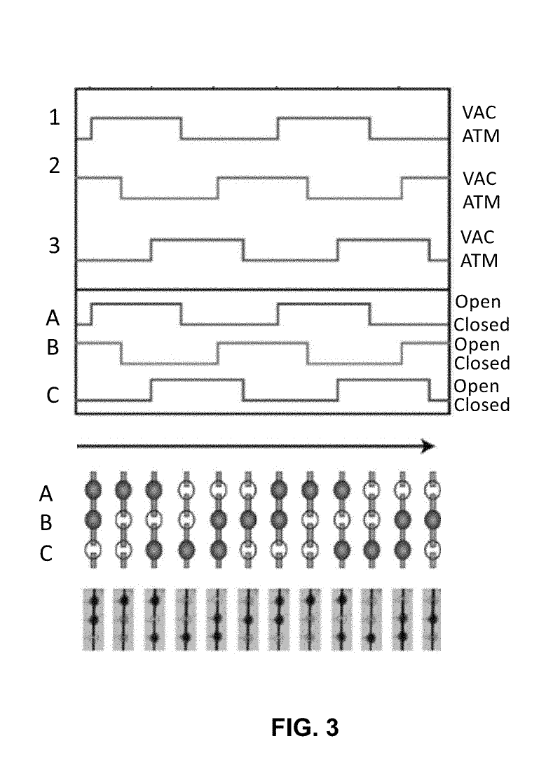

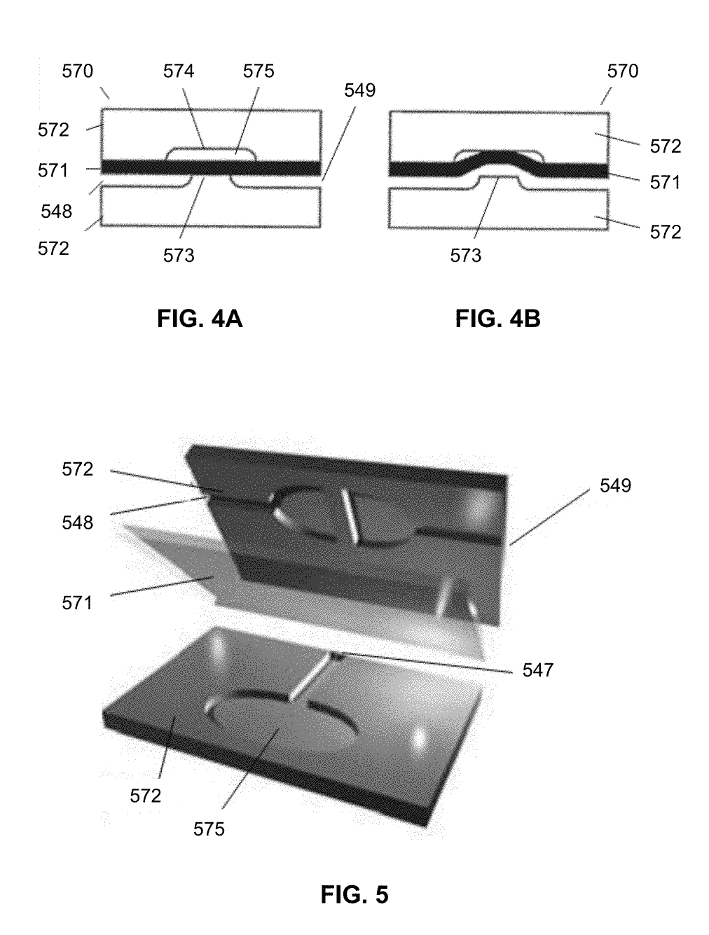

### DETAILED DESCRIPTION OF THE INVENTION

Following is a list of elements corresponding to a particular element referred to herein:

500 Multiwell stirring plate

501 Plate body

502 Well

504 Pump inlet

506 Pump outlet

508 Fluid channel

510 Pump

511 Fluid Jet

512 Convective flow

520 Pneumatic line

525 Pneumatic connection

530 Pressure source

540 Control mechanism

542 Oscillator circuit

544 Pneumatic channel

545 Logic gate

546 Pump valve

547 Valve control channel

548 Valve input channel

549 Valve output channel

550 Node

560 Pull-up resistor channel

570 Control valve

571 Membrane

572 Valve substrate

573 Valve seat

574 Chamber wall

575 Displacement chamber

600 Pump system

601 Microfluidic substrate

In one embodiment, the present invention features an integrated multiwell stirring plate (500). As a non-limiting example, the stirring plate (500) may comprise: a plate body (501); a plurality of wells (502); a plurality of pneumatic, peristaltic pumps (510); and one or more microfluidic pneumatic control mechanisms (540) configured to control the pumps (510). In some embodiments, the plurality of wells (502) may be embedded within the plate body (501). In other embodiments, each pump may comprise: a pump inlet (504); a pump outlet (506); and a fluid channel (508). In some embodiments, the pump outlet (506) may be fluidly connected with one of the wells (502). In other embodiments, the fluid channel (508) may fluidly connect the pump in line between the pump inlet (504) and the pump outlet (506). In still other embodiments, the pump (510) may be configured to pump a fluid through the fluid channel (508) and out of the pump outlet (506) so as to produce a fluid jet (511) into the well (502). In yet other embodiments, each jet (511) may be configured to impart a convective flow (512) of the fluid within the well (502). According to one embodiment, both the peristaltic pumps (510) and the control mechanisms (540) may be embedded and integrated within the plate body (501). In another embodiment the peristaltic pumps (510) are embedded and integrated within the plate body (501) and the control mechanisms (540) sit on a separate chip which is attached to the plate body (501).

In some embodiments, the jet (511) may be angled to agitate the fluid in a flow pattern. As a non-limiting example, the flow pattern may a rotational flow pattern. In other embodiments, the flow pattern may be configured for organoid culture. As a non-limiting example, the flow pattern may have a speed and direction which promotes growth of an organoid culture.

In an embodiment, the pumps (510) may be connected with the control mechanisms (540) via pneumatic lines (520). In another embodiment, each pump (510) may be configured to be coupled with a pressure source (530) via a single pneumatic connection (525) so as to be powered by a positive or negative pressure. As a non-limiting example, the negative pressure may be a vacuum pressure. In yet another embodiment, a speed of the convective flow may be directly proportional to strength of the positive or negative pressure.

According to some embodiments, each well (502) may be fluidly connected to multiple pumps (510). In other embodiments, a pump (510) may be connected to multiple wells (520). In some embodiments, a well may be fluidly connected with multiple pump outlets (506). In some other embodiments, the pump inlet (504) may be fluidly connected to the same well (502) as the pump outlet (506), and the pump (510) may be configured to recirculate the fluid in a closed loop. In still other embodiments, the pump (510) may be configured to circulate the fluid from one well (502) or reservoir to another well (502) or reservoir.

In one embodiment, the control mechanism (540) may comprise a microfluidic oscillator circuit (542). As a non-limiting example, the oscillator circuit may comprise a plurality of pneumatic channels (544); and one or more positive or negative pressure driven pneumatic inverter logic gates (545) connected in a loop by the pneumatic channels (544). In some embodiments, each logic gate (545) may exhibit a gain.

In some embodiments, each pump (510) may comprise a plurality of membrane valves (546) in line with the fluid channel (508). As a non-limiting example, each membrane valve (546) may comprise: a membrane valve control channel (547); a membrane valve input channel (548); and a membrane valve output channel (549). In one embodiment, the membrane valve input channel (548) may be fluidly connected in line with the fluid channel (508). In another embodiment, the membrane valve output channel (549) may be fluidly connected in line with the fluid channel (508). In yet another embodiment, when positive or negative pressure is applied to the membrane valve control channel (547), the membrane valve (546) may open to allow the fluid to flow from the membrane valve input channel (548) to the membrane valve output channel (549). In still another embodiment, when atmospheric pressure is applied to the membrane valve control channel (547), the membrane valve (546) may close.

According to one embodiment, each of the one or more inverter logic gates (545) may further comprise a pull-up resistor channel (560). In a further embodiment, the pull-up resistor channel (560) may comprise a long narrow channel separating the pressure source (530) from the logic gate (545). In another further embodiment, each pull-up resistor channel (560) may have a pull-up resistance that varies as a function of the length of the long narrow channel. In still another further embodiment, an oscillation frequency of the pressure oscillator circuit (542) may vary as a function of the pull-up resistance.

In an embodiment, the present invention may feature an integrated multiwell stirring plate (500). As a non-limiting example, the stirring plate (500) may comprise: a plate body (501); a plurality of wells (502); a plurality of pneumatic, peristaltic pumps (510); and one or more microfluidic pneumatic control mechanisms (540). In one embodiment, the plurality of wells (502) may be embedded within the plate body (501). In another embodiment, the plurality of pneumatic, peristaltic pumps (510) may be embedded and integrated within the plate body (501). As a non-limiting example, each pump (510) may comprise: a pump inlet (504); a pump outlet (506); a fluid channel (508); and a plurality of fluid valves (546) within the fluid channel (508). In some embodiments, the pump outlet (506) may be fluidly connected with one of the wells (502). In other embodiments, the fluid channel (508) may fluidly connect the pump in line between the pump inlet (504) and the pump outlet (506). In still other embodiments, the fluid valves (546) may be configured to move a fluid within the fluid channel (508). In one embodiment, the pump (510) may be configured to pump the fluid through the fluid channel (508) and out of the pump outlet (506) so as to produce a fluid jet into the well (502). In another embodiment, the jets (511) may be configured to impart a convective flow (512) of the fluid within the well (502). In still another embodiment, the control mechanisms (540) may be embedded and integrated within the plate body (501).

In some embodiments, each control mechanism (540) may comprise a microfluidic oscillator circuit (542) and a plurality of valve control channels (547). As a non-limiting example, the microfluidic oscillator circuit (542) may comprise an odd number of pneumatic inverter logic gates (545) connected in a closed loop; and a plurality of nodes (550), each node (550) being located between two logic gates (545) in the loop. In one embodiment, each control channel (547) may fluidly connect one of the nodes (550) with one of the fluid valves (546) such that the positive or negative pressure at the node (550) is configured to operate the valve (546). In another embodiment, the control mechanisms (540) may be configured to open and close the plurality of fluid valves (546) in a controlled manner so as to cause peristaltic pumping of the fluid within each fluid channel (508).

In one embodiment, the entire multiwell stirring plate (500) may configured to be powered and operated by a single pneumatic connection (525) to a positive or negative pressure source (530). As a non-limiting example, this configuration may allow the multiwell stirring plate (500) to be stackable. According to another embodiment, one of the control mechanisms (540) may control multiple pumps (510).

The present invention may feature a pneumatic peristaltic pump system (600). As a non-limiting example, the pump system (600) may comprise: a microfluidic substrate (601); a peristaltic pump (510), embedded and integrated within the substrate (601); and a microfluidic pneumatic control mechanism (540), embedded and integrated within the substrate (601) and fluidly connected with the pump (510). In one embodiment the pump (510) may comprise a fluid channel (508) and a plurality of pump valves (546) within the fluid channel (508). In another embodiment, the pump valves (546) may be configured to move a fluid within the fluid channel (508). In some embodiments, the microfluidic pneumatic control mechanism (540) may comprise: a microfluidic oscillator circuit (542) and a plurality of valve control channels (547). In a further embodiment, the microfluidic oscillator circuit (542) may comprise: an odd number of pneumatic inverter logic gates (545) connected in a closed loop; and a plurality of nodes (550), each node (550) being located between two logic gates (545) in the loop.

In some embodiments, each control channel (547) may fluidly connect one of the nodes (550) with one of the pump valves (546) such that positive or negative pressure at the node (550) is configured to operate the pump valve (546). In other embodiments, the control mechanism (540) may be configured to open and close the plurality of pump valves (546) in a controlled manner so as to cause peristaltic pumping to move the fluid within the fluid channel (508). In still other embodiments, the entire pump system (600) may be configured to be powered and operated by a single pneumatic connection (525) to a positive or negative pressure source (530). In yet other embodiments, a rate of the peristaltic pumping may be directly proportional to a strength of the pressure source.

In one embodiment, the pump system (600) is configured to be powered by positive pressure. In another embodiment, the pump system (600) is configured to be powered by negative pressure. To convert the negative pressure powered embodiments into positive pressure embodiments, the vacuum-powered inverter logic gates may be replaced with positive pressure-powered inverter logic gates. One main difference of the two embodiments is that while the vacuum-powered gates are closed at rest, the positive pressure-powered gates are open at rest.

In one embodiment, each logic gate (545) may comprise: a valve control channel (547); a valve input channel (548); a valve output channel (549); and a pull-up resistor channel (560). In another embodiment, the valve control channel (547) may be fluidly connected in line with the closed loop of the oscillator circuit (542). In still another embodiment, the valve input channel (548) may be fluidly connected in line with atmospheric pressure. In yet another embodiment, the valve output channel (549), may be fluidly connected in line with both the pressure source (530) and the closed loop of the oscillator circuit (542). In some embodiments, the pull-up resistor channel (560) may be fluidly connected in line between the pressure source (530) and the rest of the oscillator circuit (542).

In some embodiments, each pump valve (546) may comprise: a valve control channel (547); a valve input channel (548), fluidly connected in line with the fluid channel (508); and a valve output channel (549), fluidly connected in line with the fluid channel (508). In other embodiments, when positive or negative pressure is applied to the valve control channel (547), the pump valve (546) may open allowing the fluid to flow from the valve input channel (548) to the valve output channel (549). According to some other embodiments, when atmospheric pressure is applied to the valve control channel (547), the valve (546) may close.

According to an embodiment, each pneumatic inverter logic gate (545) may further comprise a pull-up resistor channel (560). As a non-limiting example, the pull-up resistor channel (560) may comprise a long narrow channel separating the pressure source (530) from the logic gate (545). In one embodiment, the pull-up resistor channel (560) may have a pull-up resistance that varies as a function of a length of the long narrow channel. In another embodiment, an oscillation frequency of the ring oscillator circuit (542) may vary as a function of the pull-up resistance.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. An integrated multiwell stirring plate (500) comprising:
a. a plate body (501);
b. a plurality of wells (502) embedded within the plate body (501); and
c. a plurality of pneumatic, peristaltic pumps (510), each pump comprising:
i. a pump inlet (504);
ii. a pump outlet (506), fluidly connected with one of the wells (502); and
iii. a fluid channel (508), fluidly connecting the pump in line between the pump inlet (504) and the pump outlet (506);
wherein the pump (510) is configured to pump a fluid through the fluid channel (508) and out of the pump outlet (506) so as to produce a fluid jet (511) into the well (502), and wherein each jet (511) is configured to impart a convective flow (512) of the fluid within the well (502); and
d. one or more microfluidic pneumatic control mechanisms (540) configured to control the pumps (510);
wherein both the peristaltic pumps (510) and the control mechanisms (540) are embedded and integrated within the plate body (501).

2. The multiwell stirring plate of claim 1, wherein the jet (511) is angled to agitate the fluid in a flow pattern.

3. The multiwell stirring plate of claim 2, wherein the flow pattern is a rotational flow pattern.

4. The multiwell stirring plate of claim 2, wherein the flow pattern is configured for organoid culture.

5. The multiwell stirring plate of claim 1, wherein the pumps (510) are connected with the control mechanisms (540) via pneumatic lines (520).

6. The multiwell stirring plate of claim 1, wherein each pump (510) is configured to be coupled with a pressure source (530) via a single pneumatic connection (525) so as to be powered by a positive or negative pressure.

7. The multiwell stirring plate of claim 6, wherein a speed of the convective flow is directly proportional to strength of the positive or negative pressure.

8. The multiwell stirring plate of claim 1, wherein each well (502) is fluidly connected to multiple pumps (510).

9. The multiwell stirring plate of claim 1, wherein the pump inlet (504) is fluidly connected to the same well (502) as the pump outlet (506), and wherein the pump (510) is configured to recirculate the fluid in a closed loop.

10. The multiwell stirring plate of claim 1, wherein the control mechanism (540) comprises a microfluidic oscillator circuit (542), comprising:
a. a plurality of pneumatic channels (544); and
b. one or more positive or negative pressure driven pneumatic inverter logic gates (545) connected in a loop by the pneumatic channels (544); wherein each logic gate (545) exhibits a gain.

11. The multiwell stirring plate of claim 10, wherein each pump (510) comprises a plurality of membrane valves (546) in line with the fluid channel (508), each membrane valve (546) comprising:
a. a membrane valve control channel (547);
b. a membrane valve input channel (548), fluidly connected in line with the fluid channel (508); and
c. a membrane valve output channel (549), fluidly connected in line with the fluid channel (508);
wherein when positive or negative pressure is applied to the membrane valve control channel (547), the membrane valve (546) opens allowing the fluid to flow from the membrane valve input channel (548) to the membrane valve output channel (549), and
wherein when atmospheric pressure is applied to the membrane valve control channel (547), the membrane valve (546) closes.

12. The multiwell stirring plate of claim 10, wherein each of the one or more inverter logic gates (545) further comprises a pull-up resistor channel (560),
wherein the pull-up resistor channel (560) comprises a long narrow channel separating the pressure source (530) from the logic gate (545),
wherein each pull-up resistor channel (560) has a pull-up resistance that varies as a function of the length of the long narrow channel, and
wherein an oscillation frequency of the pressure oscillator circuit (542) varies as a function of the pull-up resistance.

13. An integrated multiwell stirring plate (500) comprising:
a. a plate body (501);
b. a plurality of wells (502) embedded within the plate body (501);
c. a plurality of pneumatic, peristaltic pumps (510), embedded and integrated within the plate body (501), each pump (510) comprising:
i. a pump inlet (504);
ii. a pump outlet (506), fluidly connected with one of the wells (502);
iii. a fluid channel (508), fluidly connecting the pump in line between the pump inlet (504) and the pump outlet (506); and
iv. a plurality of fluid valves (546) within the fluid channel (508), the valves (546) configured to move a fluid within the fluid channel (508);
wherein the pump (510) is configured to pump the fluid through the fluid channel (508) and out of the pump outlet (506) so as to produce a fluid jet into the well (502), and wherein the jets (511) are configured to impart a convective flow (512) of the fluid within the well (502); and
d. one or more microfluidic pneumatic control mechanisms (540), embedded and integrated within the plate body (501), each control mechanism (540) comprising:
i. a microfluidic oscillator circuit (542) comprising:
1. an odd number of pneumatic inverter logic gates (545) connected in a closed loop; and
2. a plurality of nodes (550), each node (550) being located between two logic gates (545) in the loop; and
ii. a plurality of valve control channels (547), each control channel (547) fluidly connecting one of the nodes (550) with one of the fluid valves (546) such that the positive or negative pressure at the node (550) is configured to operate the valve (546);
wherein the control mechanisms (540) are configured to open and close the plurality of fluid valves (546) in a controlled manner so as to cause peristaltic pumping of the fluid within each fluid channel (508).

14. The multiwell stirring plate of claim 15, wherein the entire multiwell stirring plate (500) is configured to be powered and operated by a single pneumatic connection (525) to a positive or negative pressure source (530).

15. The multiwell stirring plate of claim 15, wherein one of the control mechanisms (540) controls multiple pumps (510).

16. A pneumatic peristaltic pump system (600) comprising:
a. a microfluidic substrate (601);
b. a peristaltic pump (510), embedded and integrated within the substrate (601), the pump (510) comprising:
i. a fluid channel (508);
ii. a plurality of pump valves (546) within the fluid channel (508), the pump valves (546) configured to move a fluid within the fluid channel (508);
c. a microfluidic pneumatic control mechanism (540), embedded and integrated within the substrate (601) and fluidly connected with the pump (510), the control mechanism (540) comprising:
i. a microfluidic oscillator circuit (542) comprising:
1. an odd number of pneumatic inverter logic gates (545) connected in a closed loop; and
2. a plurality of nodes (550), each node (550) being located between two logic gates (545) in the loop;
ii. a plurality of valve control channels (547), each control channel (547) fluidly connecting one of the nodes (550) with one of the pump valves (546) such that positive or negative pressure at the node (550) is configured to operate the pump valve (546);
wherein the control mechanism (540) is configured to open and close the plurality of pump valves (546) in a controlled manner so as to cause peristaltic pumping to move the fluid within the fluid channel (508), and
wherein the entire pump system (600) is configured to be powered and operated by a single pneumatic connection (525) to a positive or negative pressure source (530).

17. The pump of claim 16, wherein a rate of the peristaltic pumping is directly proportional to a strength of the pressure source.

18. The pump of claim 16, wherein each logic gate (545) comprises:
a. a valve control channel (547), fluidly connected in line with the closed loop of the oscillator circuit (542);
b. a valve input channel (548), fluidly connected in line with atmospheric pressure;
c. a valve output channel (549), fluidly connected in line with both the pressure source (530) and the closed loop of the oscillator circuit (542); and
d. a pull-up resistor channel (560), fluidly connected in line between the pressure source (530) and the rest of the oscillator circuit (542).

19. The pump of claim 16, wherein each pump valve (546) comprises:
a. a valve control channel (547);
b. a valve input channel (548), fluidly connected in line with the fluid channel (508); and
c. a valve output channel (549), fluidly connected in line with the fluid channel (508);
wherein when positive or negative pressure is applied to the valve control channel (547), the pump valve (546) opens allowing the fluid to flow from the valve input channel (548) to the valve output channel (549), and
wherein when atmospheric pressure is applied to the valve control channel (547), the valve (546) closes.

20. The pump of claim 19, wherein each pneumatic inverter logic gate (545) further comprises a pull-up resistor channel (560) comprising a long narrow channel separating the pressure source (530) from the logic gate (545),
wherein the pull-up resistor channel (560) has a pull-up resistance that varies as a function of a length of the long narrow channel, and
wherein an oscillation frequency of the ring oscillator circuit (542) varies as a function of the pull-up resistance.
